SEQUENCE LISTING (100 sequences)

SEQ ID NO: 1 - DNA, 120 bp, synthetic construct
  source 1..120: mol_type = other DNA
acagatctgggaggtatgcagaatgaagtttcagggcaaaagagtacactncttcatttttaaagttagaagctggaaatgcattctgccaaattttagttgtagctcaagcatatttca

SEQ ID NO: 2 - DNA, 120 bp, synthetic construct
  source 1..120: mol_type = other DNA
gcattgtctccatggcacttcctccaggaaggagattcacagctctttatngacttttgattgtagattcataggaatacatattagcatatggatctattattgtaacctgtggaaatg

SEQ ID NO: 3 - DNA, 120 bp, synthetic construct
  source 1..120: mol_type = other DNA
ctctgcagcagcaccatgtccctgtgaaggagtttctgaggcacagcaatntgggttgcaccatgacgtggccacatggatgtgctcagtgccacccccacagcaaagtgatgctagctg

SEQ ID NO: 4 - DNA, 120 bp, synthetic construct
  source 1..120: mol_type = other DNA
ggatagctgcaggaaaagctgactgtcaccattacctgtgggattatcctnagtaacccaccacgaatagaagttaaaatagcctcccacctgtctgtagaattgagattccccttcaaa

SEQ ID NO: 5 - DNA, 120 bp, synthetic construct
  source 1..120: mol_type = other DNA
aatggcaagacattacaggagataacagtgcagtacgatgcgatgtgatancggtgtgaaaagaaaccaaggaatggagtaatgggaagaaaaataaatcaggtgacaaacgtttaagca

SEQ ID NO: 6 - DNA, 120 bp, synthetic construct
  source 1..120: mol_type = other DNA
ccgctgccctgcgagccgggagcaccccgagcacacggaggaggacgagcngagccatccccgcggtcctttgcatacggggctcgagggcttctcctagcatgctgagccccggtgagg

SEQ ID NO: 7 - DNA, 120 bp, synthetic construct
  source 1..120: mol_type = other DNA
agaaaaatatttaccttctgactttttctttttggaattcaccatcctcancacgtgagtcagcgttcagctcttcagattcctctgactcagctgcctggtagcaggaacacttgaaat

SEQ ID NO: 8 - DNA, 120 bp, synthetic construct
  source 1..120: mol_type = other DNA
tttagaaactatcctgaattaaatatttatgtttaagcttttacaaggcantctgaataagtcttttgggttttttccacagcagccttaactgtacttagccgtataggagagttcgat

SEQ ID NO: 9 - DNA, 120 bp, synthetic construct
  source 1..120: mol_type = other DNA
ttgtcacagcactgatattctgtgaccttccacgttagttatgatggagantgaaatcattgctgtgtatttgatggctcctttgtctggcatggtgacaactgcaacagactgtcaata

SEQ ID NO: 10 - DNA, 120 bp, synthetic construct
  source 1..120: mol_type = other DNA
aaagtaacctttcctccaaggctgcttataacaaacaaatgaaaataaagnagtgggagtcagtaactgttaaatcaccccaacgtgcaatgctaatattttggttttgccttacataaa

SEQ ID NO: 11 - DNA, 120 bp, synthetic construct
  source 1..120: mol_type = other DNA
gtttgcaccggggctgagcatcagctctctgatcctagaggcaggagcctntactctccgagctaaacagcctgctgcagttgcctgtcttctagggtgtctttagcctctccgtcttcc

SEQ ID NO: 12 - DNA, 120 bp, synthetic construct
  source 1..120: mol_type = other DNA
ggggtcacatggaggtgtcggcgaggctctgcgcggtgccgcggccgttcngggatgcgccgttctcctgcgccccgttggcgctcggcttctcctgctgtgcagggctgccctggctgc

SEQ ID NO: 13 - DNA, 120 bp, synthetic construct
  source 1..120: mol_type = other DNA
tcagtgatggccctcattcaagagtccagaggaataaggttggagataacncactcagtgccaaccctctgccacccaaaaggctggggggtcctctgttggatgttggcaggaattgat

SEQ ID NO: 14 - DNA, 120 bp, synthetic construct
  source 1..120: mol_type = other DNA
tatattgtgttagtcttgggggggaaaccgatttccttttcgttcaccttntcacaaacaaaagatacttcagtaggaagaggagcaatattgcagtgctgggctgtgactgcacagggg

SEQ ID NO: 15 - DNA, 120 bp, synthetic construct
  source 1..120: mol_type = other DNA
atattatcttatataatcctagataaagcagaaagataaggtatgcctagnatgcttatccttgggcagagccaggactataacaggggaagattcagctctgaaaacacagctcccaga

SEQ ID NO: 16 - DNA, 120 bp, synthetic construct
  source 1..120: mol_type = other DNA
ttcgatctctctgaattccttgagcataagaaaaattgcactaaaaatccncctgtcctaatcatgaatgacagtgagggagcagtccctcctgaaggcttctccgaagctgctgttggg

SEQ ID NO: 17 - DNA, 120 bp, synthetic construct
  source 1..120: mol_type = other DNA
tcgtgcacgcatgcctgcaggtttgcgagcccttagcaaccagctccctgngcctgcacgctcgtgtgccttgcaaatggagctgcaccatgtgcaccgggctgcattttcagacaaaga

SEQ ID NO: 18 - DNA, 120 bp, synthetic construct
  source 1..120: mol_type = other DNA
tgactgatttgcccacggtgatgcagaatgcctctggtaaaatggcagctngcctgagggatcccaggctgccaggaccccgagccacatcctgagtgcaggcacaggggtagcaatgag

SEQ ID NO: 19 - DNA, 120 bp, synthetic construct
  source 1..120: mol_type = other DNA
ctttctggcctctcccaggaatcctttcctacaacccctaagtcagcaccnggtcagagacaggggaaaacttgggaagcacacacactttgtggtttcccttgaacctttgaatggact

SEQ ID NO: 20 - DNA, 120 bp, synthetic construct
  source 1..120: mol_type = other DNA
tcctgctgctaatatagcaacaaagagaagacctgatcttgaaagcagtancttgttatcagaaagcaacatgtgttactgcaaacagcaaatgtatggaacaatgtgcctctatctgcc

SEQ ID NO: 21 - DNA, 120 bp, synthetic construct
  source 1..120: mol_type = other DNA
gtcaggcattcacaaaacaaaagattaaaataataggaatgctgtatagcngacaaattactctgctgagctgcctgtagtaacagaggcactgacccctgaaggacttcacagagccgt

SEQ ID NO: 22 - DNA, 120 bp, synthetic construct
  source 1..120: mol_type = other DNA
aatcagctgcaattcttcagctccatttattctacccttcattttaaaacnacaggtacacagcaggcatcagcagggaggttgcacagcaggtagaaatatcaaaagttttgttgtgct

SEQ ID NO: 23 - DNA, 120 bp, synthetic construct
  source 1..120: mol_type = other DNA
gaaatttagatgccatgtcccctgggcagcaaataactgcaggggcttaanagcatccctggcatcctgtgcagagagggtagggcacaaggaaaccacagagggtactgacaaaagatt

SEQ ID NO: 24 - DNA, 120 bp, synthetic construct
  source 1..120: mol_type = other DNA
tcctcatttccttcagagccacctgaaggagcaggaagctgaatagacttntcatggaagacagatcattccactagctgttagttggaccagattctgttgttatattcaccttctcgt

SEQ ID NO: 25 - DNA, 120 bp, synthetic construct
  source 1..120: mol_type = other DNA
cagaggtgaacgctgcgccctcctgttcgcaaacagctgcagacagaaacntgcagccaatccctgcactgccctgcctgatgaataggggacagatttgcgctgattctgctctcacgc

SEQ ID NO: 26 - DNA, 47 bp, synthetic construct
  source 1..47: mol_type = other DNA
gaaggtgaccaagttcatgctaagtttcagggcaaaagagtacacta

SEQ ID NO: 27 - DNA, 47 bp, synthetic construct
  source 1..47: mol_type = other DNA
gaaggtcggagtcaacggattaagtttcagggcaaaagagtacactt

SEQ ID NO: 28 - DNA, 32 bp, synthetic construct
  source 1..32: mol_type = other DNA
gaatgcatttccagcttctaactttaaaaatg

SEQ ID NO: 29 - DNA, 47 bp, synthetic construct
  source 1..47: mol_type = other DNA
gaaggtgaccaagttcatgctaggaaggagattcacagctctttatt

SEQ ID NO: 30 - DNA, 46 bp, synthetic construct
  source 1..46: mol_type = other DNA
gaaggtcggagtcaacggattggaaggagattcacagctctttatc

SEQ ID NO: 31 - DNA, 34 bp, synthetic construct
  source 1..34: mol_type = other DNA
atatgctaatatgtattcctatgaatctacaatc

SEQ ID NO: 32 - DNA, 43 bp, synthetic construct
  source 1..43: mol_type = other DNA
gaaggtgaccaagttcatgctgagtttctgaggcacagcaatc

SEQ ID NO: 33 - DNA, 43 bp, synthetic construct
  source 1..43: mol_type = other DNA
gaaggtcggagtcaacggattgagtttctgaggcacagcaatg

SEQ ID NO: 34 - DNA, 23 bp, synthetic construct
  source 1..23: mol_type = other DNA
cacatccatgtggccacgtcatg

SEQ ID NO: 35 - DNA, 44 bp, synthetic construct
  source 1..44: mol_type = other DNA
gaaggtgaccaagttcatgctcttctattcgtggtgggttactc

SEQ ID NO: 36 - DNA, 47 bp, synthetic construct
  source 1..47: mol_type = other DNA
gaaggtcggagtcaacggatttaacttctattcgtggtgggttactt

SEQ ID NO: 37 - DNA, 25 bp, synthetic construct
  source 1..25: mol_type = other DNA
aagctgactgtcaccattacctgtg

SEQ ID NO: 38 - DNA, 44 bp, synthetic construct
  source 1..44: mol_type = other DNA
gaaggtgaccaagttcatgcttccttggtttcttttcacaccgg

SEQ ID NO: 39 - DNA, 46 bp, synthetic construct
  source 1..46: mol_type = other DNA
gaaggtcggagtcaacggattattccttggtttcttttcacaccga

SEQ ID NO: 40 - DNA, 25 bp, synthetic construct
  source 1..25: mol_type = other DNA
aggagataacagtgcagtacgatgc

SEQ ID NO: 41 - DNA, 39 bp, synthetic construct
  source 1..39: mol_type = other DNA
gaaggtgaccaagttcatgctcacggaggaggacgagcg

SEQ ID NO: 42 - DNA, 41 bp, synthetic construct
  source 1..41: mol_type = other DNA
gaaggtcggagtcaacggattcacacggaggaggacgagca

SEQ ID NO: 43 - DNA, 22 bp, synthetic construct
  source 1..22: mol_type = other DNA
tcgagccccgtatgcaaaggac

SEQ ID NO: 44 - DNA, 47 bp, synthetic construct
  source 1..47: mol_type = other DNA
gaaggtgaccaagttcatgctttctttttggaattcaccatcctcag

SEQ ID NO: 45 - DNA, 48 bp, synthetic construct
  source 1..48: mol_type = other DNA
gaaggtcggagtcaacggatttttctttttggaattcaccatcctcaa

SEQ ID NO: 46 - DNA, 25 bp, synthetic construct
  source 1..25: mol_type = other DNA
aatctgaagagctgaacgctgactc

SEQ ID NO: 47 - DNA, 55 bp, synthetic construct
  source 1..55: mol_type = other DNA
gaaggtgaccaagttcatgctattaaatatttatgtttaagcttttacaaggcaa

SEQ ID NO: 48 - DNA, 55 bp, synthetic construct
  source 1..55: mol_type = other DNA
gaaggtcggagtcaacggattattaaatatttatgtttaagcttttacaaggcat

SEQ ID NO: 49 - DNA, 27 bp, synthetic construct
  source 1..27: mol_type = other DNA
ctaagtacagttaaggctgctgtggaa

SEQ ID NO: 50 - DNA, 46 bp, synthetic construct
  source 1..46: mol_type = other DNA
gaaggtgaccaagttcatgctcttccacgttagttatgatggagag

SEQ ID NO: 51 - DNA, 46 bp, synthetic construct
  source 1..46: mol_type = other DNA
gaaggtcggagtcaacggattcttccacgttagttatgatggagaa

SEQ ID NO: 52 - DNA, 26 bp, synthetic construct
  source 1..26: mol_type = other DNA
gacaaaggagccatcaaatacacagc

SEQ ID NO: 53 - DNA, 46 bp, synthetic construct
  source 1..46: mol_type = other DNA
gaaggtgaccaagttcatgctatttaacagttactgactcccactc

SEQ ID NO: 54 - DNA, 47 bp, synthetic construct
  source 1..47: mol_type = other DNA
gaaggtcggagtcaacggattgatttaacagttactgactcccactt

SEQ ID NO: 55 - DNA, 28 bp, synthetic construct
  source 1..28: mol_type = other DNA
ctccaaggctgcttataacaaacaaatg

SEQ ID NO: 56 - DNA, 43 bp, synthetic construct
  source 1..43: mol_type = other DNA
gaaggtgaccaagttcatgctggctgtttagctcggagagtag

SEQ ID NO: 57 - DNA, 43 bp, synthetic construct
  source 1..43: mol_type = other DNA
gaaggtcggagtcaacggattggctgtttagctcggagagtac

SEQ ID NO: 58 - DNA, 23 bp, synthetic construct
  source 1..23: mol_type = other DNA
cagctctctgatcctagaggcag

SEQ ID NO: 59 - DNA, 39 bp, synthetic construct
  source 1..39: mol_type = other DNA
gaaggtgaccaagttcatgctggagaacggcgcatcccg

SEQ ID NO: 60 - DNA, 41 bp, synthetic construct
  source 1..41: mol_type = other DNA
gaaggtcggagtcaacggattcaggagaacggcgcatccca

SEQ ID NO: 61 - DNA, 20 bp, synthetic construct
  source 1..20: mol_type = other DNA
aggtgtcggcgaggctctgc

SEQ ID NO: 62 - DNA, 46 bp, synthetic construct
  source 1..46: mol_type = other DNA
gaaggtgaccaagttcatgctcagaggaataaggttggagataaca

SEQ ID NO: 63 - DNA, 46 bp, synthetic construct
  source 1..46: mol_type = other DNA
gaaggtcggagtcaacggattcagaggaataaggttggagataacg

SEQ ID NO: 64 - DNA, 22 bp, synthetic construct
  source 1..22: mol_type = other DNA
ccttttgggtggcagagggttg

SEQ ID NO: 65 - DNA, 47 bp, synthetic construct
  source 1..47: mol_type = other DNA
gaaggtgaccaagttcatgctaaccgatttccttttcgttcacctta

SEQ ID NO: 66 - DNA, 45 bp, synthetic construct
  source 1..45: mol_type = other DNA
gaaggtcggagtcaacggattccgatttccttttcgttcaccttc

SEQ ID NO: 67 - DNA, 29 bp, synthetic construct
  source 1..29: mol_type = other DNA
cctcttcctactgaagtatcttttgtttg

SEQ ID NO: 68 - DNA, 47 bp, synthetic construct
  source 1..47: mol_type = other DNA
gaaggtgaccaagttcatgctaagcagaaagataaggtatgcctaga

SEQ ID NO: 69 - DNA, 45 bp, synthetic construct
  source 1..45: mol_type = other DNA
gaaggtcggagtcaacggattgcagaaagataaggtatgcctagg

SEQ ID NO: 70 - DNA, 23 bp, synthetic construct
  source 1..23: mol_type = other DNA
ctgttatagtcctggctctgccc

SEQ ID NO: 71 - DNA, 46 bp, synthetic construct
  source 1..46: mol_type = other DNA
gaaggtgaccaagttcatgctactgtcattcatgattaggacaggt

SEQ ID NO: 72 - DNA, 45 bp, synthetic construct
  source 1..45: mol_type = other DNA
gaaggtcggagtcaacggattctgtcattcatgattaggacaggc

SEQ ID NO: 73 - DNA, 31 bp, synthetic construct
  source 1..31: mol_type = other DNA
tctctgaattccttgagcataagaaaaattg

SEQ ID NO: 74 - DNA, 41 bp, synthetic construct
  source 1..41: mol_type = other DNA
gaaggtgaccaagttcatgctcttagcaaccagctccctgc

SEQ ID NO: 75 - DNA, 42 bp, synthetic construct
  source 1..42: mol_type = other DNA
gaaggtcggagtcaacggattccttagcaaccagctccctgt

SEQ ID NO: 76 - DNA, 23 bp, synthetic construct
  source 1..23: mol_type = other DNA
ctccatttgcaaggcacacgagc

SEQ ID NO: 77 - DNA, 43 bp, synthetic construct
  source 1..43: mol_type = other DNA
gaaggtgaccaagttcatgctgcctctggtaaaatggcagctg

SEQ ID NO: 78 - DNA, 44 bp, synthetic construct
  source 1..44: mol_type = other DNA
gaaggtcggagtcaacggatttgcctctggtaaaatggcagcta

SEQ ID NO: 79 - DNA, 20 bp, synthetic construct
  source 1..20: mol_type = other DNA
ggtcctggcagcctgggatc

SEQ ID NO: 80 - DNA, 43 bp, synthetic construct
  source 1..43: mol_type = other DNA
gaaggtgaccaagttcatgcttacaacccctaagtcagcacca

SEQ ID NO: 81 - DNA, 42 bp, synthetic construct
  source 1..42: mol_type = other DNA
gaaggtcggagtcaacggattacaacccctaagtcagcaccg

SEQ ID NO: 82 - DNA, 25 bp, synthetic construct
  source 1..25: mol_type = other DNA
agtgtgtgtgcttcccaagttttcc

SEQ ID NO: 83 - DNA, 46 bp, synthetic construct
  source 1..46: mol_type = other DNA
gaaggtgaccaagttcatgctgaagacctgatcttgaaagcagtac

SEQ ID NO: 84 - DNA, 47 bp, synthetic construct
  source 1..47: mol_type = other DNA
gaaggtcggagtcaacggattagaagacctgatcttgaaagcagtat

SEQ ID NO: 85 - DNA, 28 bp, synthetic construct
  source 1..28: mol_type = other DNA
tgtttgcagtaacacatgttgctttctg

SEQ ID NO: 86 - DNA, 45 bp, synthetic construct
  source 1..45: mol_type = other DNA
gaaggtgaccaagttcatgctgcagctcagcagagtaatttgtct

SEQ ID NO: 87 - DNA, 44 bp, synthetic construct
  source 1..44: mol_type = other DNA
gaaggtcggagtcaacggattcagctcagcagagtaatttgtcc

SEQ ID NO: 88 - DNA, 34 bp, synthetic construct
  source 1..34: mol_type = other DNA
cacaaaacaaaagattaaaataataggaatgctg

SEQ ID NO: 89 - DNA, 43 bp, synthetic construct
  source 1..43: mol_type = other DNA
gaaggtgaccaagttcatgcttgatgcctgctgtgtacctgta

SEQ ID NO: 90 - DNA, 42 bp, synthetic construct
  source 1..42: mol_type = other DNA
gaaggtcggagtcaacggattgatgcctgctgtgtacctgtg

SEQ ID NO: 91 - DNA, 28 bp, synthetic construct
  source 1..28: mol_type = other DNA
ttcttcagctccatttattctacccttc

SEQ ID NO: 92 - DNA, 44 bp, synthetic construct
  source 1..44: mol_type = other DNA
gaaggtgaccaagttcatgctgcaaataactgcaggggcttaag

SEQ ID NO: 93 - DNA, 46 bp, synthetic construct
  source 1..46: mol_type = other DNA
gaaggtcggagtcaacggattcagcaaataactgcaggggcttaaa

SEQ ID NO: 94 - DNA, 22 bp, synthetic construct
  source 1..22: mol_type = other DNA
taccctctctgcacaggatgcc

SEQ ID NO: 95 - DNA, 45 bp, synthetic construct
  source 1..45: mol_type = other DNA
gaaggtgaccaagttcatgctgtggaatgatctgtcttccatgac

SEQ ID NO: 96 - DNA, 47 bp, synthetic construct
  source 1..47: mol_type = other DNA
gaaggtcggagtcaacggatttagtggaatgatctgtcttccatgat

SEQ ID NO: 97 - DNA, 25 bp, synthetic construct
  source 1..25: mol_type = other DNA
acctgaaggagcaggaagctgaata

SEQ ID NO: 98 - DNA, 43 bp, synthetic construct
  source 1..43: mol_type = other DNA
gaaggtgaccaagttcatgctcaaacagctgcagacagaaacc

SEQ ID NO: 99 - DNA, 44 bp, synthetic construct
  source 1..44: mol_type = other DNA
gaaggtcggagtcaacggattgcaaacagctgcagacagaaaca

SEQ ID NO: 100 - DNA, 20 bp, synthetic construct
  source 1..20: mol_type = other DNA
tcaggcagggcagtgcaggg

(19) United States
(12) Patent Application Publication
(10) Pub. No.: US 2025/0115966 A1
(43) Pub. Date: Apr. 10, 2025

(54) SINGLE NUCLEOTIDE POLYMORPHISM MOLECULAR MARKER COMBINATION FOR IDENTIFYING ARBOR ACRES BROILER, DETECTION KIT AND APPLICATION THEREOF

(71) Applicant: Henan Agricultural University, Zhengzhou (CN)

(72) Inventors: Xiangtao Kang, Zhengzhou (CN); Zhuanjian Li, Zhengzhou (CN); Yanxing Wang, Zhengzhou (CN); Tong Li, Zhengzhou (CN); Yadong Tian, Zhengzhou (CN); Ruili Han, Zhengzhou (CN); Hong Li, Zhengzhou (CN); Xiaojun Liu, Zhengzhou (CN); Weihua Tian, Zhengzhou (CN); Ruirui Jiang, Zhengzhou (CN); Guoxi Li, Zhengzhou (CN); Wenting Li, Zhengzhou (CN); Fengbin Yan, Zhengzhou (CN); Yanbin Wang, Zhengzhou (CN)

(21) Appl. No.: 18/762,681

(22) Filed: Jul. 3, 2024

Related U.S. Application Data
(63) Continuation of application No. PCT/CN2024/097035, filed on Jun. 3, 2024.

(30) Foreign Application Priority Data
Oct. 9, 2023 (CN) 202311300049.4

(51) Int. Cl. C12Q 1/6888; C12Q 1/6844

(52) U.S. Cl. CPC C12Q 1/6888; C12Q 1/6844; C12Q 2600/124; C12Q 2600/156

(57) ABSTRACT

A SNP molecular marker combination for identifying an AA broiler includes 25 SNP molecular markers, SNP sites of the SNP molecular markers 1 to 25 are located at a 51^{st} position of each of nucleotide sequences shown in SEQ ID NO: 1 to 25. The SNP molecular marker combination for identifying the AA broiler in the disclosure has apparent species specificity of the AA broiler, is a unique mutation and importance determining site for the AA broiler, and can quickly identify authenticity of the AA broiler with less genotype information, providing new technical references for the identification, conservation, and genetic breeding of chicken breeds in the future.

### CROSS-REFERENCE TO RELATED APPLICATIONS

This application is a continuation of International Patent Application No. PCT/CN2024/097035, filed on Jun. 3, 2024, which claims the priority of Chinese Patent Application No. CN202311300049.4, filed on Oct. 9, 2023, both of which are herein incorporated by reference in their entirety.

### TECHNICAL FIELD

The disclosure relates to the field of biotechnologies, and more particularly to a single nucleotide polymorphism (SNP) molecular marker combination for identifying an arbor acres (AA) broiler, a detection kit and an application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 24058TBYX-USP1-MF-2024-0057-SL.xml. The XML file is 93,074 bytes; is created on Jun. 28, 2024; and is being submitted electronically via patent center.

### BACKGROUND

SNP refers to DNA sequence polymorphism caused by variation of a single nucleotide at a genomic level. SNP gradually becomes a new generation of molecular markers and is widely used in biology, agriculture, medicine, and biological evolution, due to its advantages of large quantity and stable inheritance. Molecular marker technology can quickly, accurately, and efficiently identify breeds, which is of great significance for ensuring correctness of the breeds in poultry production. With the continuous development of poultry breeding and molecular genetics, researchers find that classifying target populations at a genetic level can provide important information for preservation and commercial utilization of the breeds. In addition, an optimal combination of SNP markers can produce stable results in validation studies analyzing unknown samples.

An AA broiler is a four-line crossbred white-feathered broiler, belonging to a fast-growing breed. The AA broiler has advantages of fast growth rate, strong adaptability, high feed conversion rate, neat development, full chest and leg muscles, and good carcass quality. At present, poultry breed identification in the related art is mainly based on traditional appearance identification only such as body size, feather color, shank color and crown shape, which is inevitably affected by environmental and other subjective factors. Moreover, chicks with unclear breed characteristics or meat products already on the market cannot be identified through morphological characteristics. Accurate identification of poultry strains at the genetic level is an effective means to solve this problem. Therefore, exploring characteristic markers of different strains is currently an urgent problem to be solved.

### SUMMARY

A first purpose of the disclosure is to provide a SNP molecular marker combination for identifying an AA broiler to solve problem of no SNP molecular marker used in identifying the AA broiler in the related art.

A second purpose of the disclosure is to provide a detection kit to solve problem that characteristic SNP molecular markers of the AA broiler cannot be detected in the related art.

A third purpose of the disclosure is to provide an application method of the SNP molecular marker combination or the detection kit to solve problem that a broiler sample to be tested cannot be accurately identified whether it is the AA broiler based on morphology in the related art.

To solve the above problems, the technical solution of the SNP molecular marker combination for identifying the AA broiler of the disclosure is below.

The SNP molecular marker combination for identifying the AA broiler includes 25 SNP molecular markers, SNP sites of the SNP molecular markers 1 to 25 are located at a 51^{st} position of each of nucleotide sequences shown in SEQ ID NO: 1 to 25.

The above technical solution has below beneficial effects: in the disclosure, whole genome sequencing data of 30 individuals of AA broilers is obtained through whole genome resequencing. By comparing and analyzing whole genome sequencing data of 336 individuals from 27 other publicly available chicken breeds, the whole genome sequencing data of the 30 individuals of the AA broilers undergoes a combination optimization to obtain 25 distinct SNP sites between AA broilers and non-AA broilers, the 25 distinct SNP sites form the SNP molecular marker combination for identifying the AA broiler. The SNP molecular marker combination for identifying the AA broiler in the disclosure has apparent species specificity of the AA broiler, and can quickly identify authenticity of the AA broiler with less genotype information, providing new technical references for the identification, conservation, and genetic breeding of chicken breeds in the future.

To achieve the above purposes, the technical solution of the detection kit of the disclosure is below.

The detection kit includes a polymerase chain reaction (PCR) primer set for detecting genotypes of the SNP molecular marker combination for identifying the AA broiler.

The above technical solution has below beneficial effects: the PCR primer set for detecting the SNP molecular marker combination genotypes is designed and synthesized, after PCR amplification, the PCR primer set with means such as gene sequencing can quickly and accurately obtain genotype information of SNP molecular markers, laying foundation for subsequent analysis and verification.

In an embodiment, the PCR primer set is a competitive allele specific PCR (KASP) primer set.

The above technical solution has below beneficial effects: KSAP is an effective method for SNP typing and detecting insertion-deletions (Indels) by using specific matching of primer end bases. The KSAP does not require synthesis of specific fluorescent primers for each SNP site. It is based on its unique amplification refractory mutation (ARM)-PCR principle, allowing all site detections to be amplified using universal fluorescent primers, achieving gene typing and detection with advantages of fast, accurate, and high-throughput.

In an embodiment, the KASP primer set includes a first primer set to a twenty-fifth primer set correspondingly detecting the SNP molecular markers 1 to 25, and nucleotide sequences of the first primer set to the twenty-fifth primer set are shown as SEQ ID NO: 26 to 100.

In an embodiment, the detection kit further includes a KASP reaction buffer, deoxyribonucleic acid (DNA) polymerase and deoxy-ribonucleoside triphosphates (dNTPs).

To achieve the above purposes, the technical solution of the application method of the detection kit or the SNP molecular marker combination is below.

The detection kit or the SNP molecular marker combination is applied in identification of AA broiler germplasm resources.

The above technical solution has below beneficial effects: in response to the SNP molecular marker combination for identifying the AA broiler, the genotypes of the SNP molecular markers are detected, and rapid and accurate identification of whether a sample to be tested is the AA broiler is achieved with a blind test accuracy rate as high as 99.47%, filling a gap where there is currently no method for identifying the AA broiler at a genetic level. It can be used for traceability identification and protection of the AA broiler germplasm resources, and is of great significance for promoting the positive development of AA broiler germplasm resources.

In an embodiment, the genotypes of the SNP molecular markers 1 to 25 in the sample to be tested are detected, when the genotypes of the SNP molecular markers 1 to 25 of the sample to be tested match genotypes (i.e., target genotypes) shown in Table 1, the sample to be tested is the AA broiler.

TABLE 1

| SNP | |
|---|---|
| molecular | |
| marker | Genotype |
| 1 | TT |
| 2 | CC |
| 3 | GG |
| 4 | AA |
| 5 | TT |
| 6 | AA |
| 7 | AA |
| 8 | AA |
| 9 | GG |
| 10 | GG |
| 11 | GG or |
| | CG |
| 12 | TT |
| 13 | AA or |
| | AG |
| 14 | AA |
| 15 | GA or |
| | AA |
| 16 | GG |
| 17 | TT |
| 18 | AA |
| 19 | GG |
| 20 | CC |
| 21 | AA |
| 22 | CC or |
| | TC |
| 23 | AG or |
| | AA |
| 24 | AA |
| 25 | CC |

In an embodiment, the step that the genotypes of the SNP molecular markers 1 to 25 in the sample to be tested are detected includes: PCR amplification reaction is performed by using the detection kit with extracted DNA of the sample to be tested as a template to obtain fluorescence signals for genotyping.

### DETAILED DESCRIPTION OF EMBODIMENTS

The purpose, technical solution, and beneficial effects of the disclosure are further explained in conjunction with embodiments. The described embodiments are helpful for those skilled in the art to better understand the disclosure and does not constitute a limitation on the disclosure. Unless otherwise specified, reagents and instruments etc., used in the embodiments are commercially available.

### Embodiment 1

### A SNP Molecular Marker Combination for Identifying an AA Broiler

The SNP molecular marker combination for identifying the AA broiler in the embodiment 1 includes 25 SNP molecular markers, SNP sites of the SNP molecular markers 1 to 25 are located at a 51^{st} position of each of nucleotide sequences shown in SEQ ID NO: 1 to 25.

### A Detection Kit

The detection kit includes a KASP primer set for detecting genotypes of the SNP molecular marker combination for identifying the AA broiler. The KASP primer set includes a first primer set to a twenty-fifth primer set correspondingly detecting the SNP molecular markers 1 to 25, and nucleotide sequences of the first primer set to the twenty-fifth primer set are shown as SEQ ID NO: 26 to 100. Specific correspondence between the SNP molecular markers and the primer sets is shown in Table 2

TABLE 2

| SNP | | | |
|---|---|---|---|
| molecular | | | |
| marker | Primer X | Primer Y | Primer C |
| 1 | SEQ ID NO: | SEQ ID NO: | SEQ ID NO: |
| | 26 | 27 | 28 |
| 2 | SEQ ID NO: | SEQ ID NO: | SEQ ID NO: |
| | 29 | 30 | 31 |
| 3 | SEQ ID NO: | SEQ ID NO: | SEQ ID NO: |
| | 32 | 33 | 34 |
| 4 | SEQ ID NO: | SEQ ID NO: | SEQ ID NO: |
| | 35 | 36 | 37 |
| 5 | SEQ ID NO: | SEQ ID NO: | SEQ ID NO: |
| | 38 | 39 | 40 |
| 6 | SEQ ID NO: | SEQ ID NO: | SEQ ID NO: |
| | 41 | 42 | 43 |
| 7 | SEQ ID NO: | SEQ ID NO: | SEQ ID NO: |
| | 44 | 45 | 46 |
| 8 | SEQ ID NO: | SEQ ID NO: | SEQ ID NO: |
| | 47 | 48 | 49 |
| 9 | SEQ ID NO: | SEQ ID NO: | SEQ ID NO: |
| | 50 | 51 | 52 |
| 10 | SEQ ID NO: | SEQ ID NO: | SEQ ID NO: |
| | 53 | 54 | 55 |
| 11 | SEQ ID NO: | SEQ ID NO: | SEQ ID NO: |
| | 56 | 57 | 58 |
| 12 | SEQ ID NO: | SEQ ID NO: | SEQ ID NO: |
| | 59 | 60 | 61 |
| 13 | SEQ ID NO: | SEQ ID NO: | SEQ ID NO: |
| | 62 | 63 | 64 |
| 14 | SEQ ID NO: | SEQ ID NO: | SEQ ID NO: |
| | 65 | 66 | 67 |
| 15 | SEQ ID NO: | SEQ ID NO: | SEQ ID NO: |
| | 68 | 69 | 70 |
| 16 | SEQ ID NO: | SEQ ID NO: | SEQ ID NO: |
| | 71 | 72 | 73 |
| 17 | SEQ ID NO: | SEQ ID NO: | SEQ ID NO: |
| | 74 | 75 | 76 |
| 18 | SEQ ID NO: | SEQ ID NO: | SEQ ID NO: |
| | 77 | 78 | 79 |
| 19 | SEQ ID NO: | SEQ ID NO: | SEQ ID NO: |
| | 80 | 81 | 82 |
| 20 | SEQ ID NO: | SEQ ID NO: | SEQ ID NO: |
| | 83 | 84 | 85 |
| 21 | SEQ ID NO: | SEQ ID NO: | SEQ ID NO: |
| | 86 | 87 | 88 |
| 22 | SEQ ID NO: | SEQ ID NO: | SEQ ID NO: |
| | 89 | 90 | 91 |
| 23 | SEQ ID NO: | SEQ ID NO: | SEQ ID NO: |
| | 92 | 93 | 94 |
| 24 | SEQ ID NO: | SEQ ID NO: | SEQ ID NO: |
| | 95 | 96 | 97 |
| 25 | SEQ ID NO: | SEQ ID NO: | SEQ ID NO: |
| | 98 | 99 | 100 |

### An Application Method of the Detection Kit or the SNP Molecular Marker Combination in Identification of AA Broiler Germplasm Resources

The application method of the detection kit or the SNP molecular marker combination in identification of AA broiler germplasm resources includes following steps.
(1) Genomic DNA of a sample to be tested is extracted to obtain extracted DNA.
(2) PCR amplification: the extracted DNA in the step (1) is used as a template to perform the PCR amplification with KASP primers as shown by the nucleotide sequences shown as SEQ ID NO: 26 to 100 to obtain fluorescence signals.
(3) Fluorescence signal acquisition and genotyping: SNP typing is performed based on a fluorescence signal ratio. When the genotypes of the 25 SNP molecular markers in the sample to be tested match the genotypes shown in the Table 1, the sample to be tested is an AA broiler.

### Experimental Embodiment 1

### Screening of SNP Molecular Marker Combination for Identification of AA Broiler

In the experimental embodiment, whole-genome resequencing is used to obtain whole-genome sequencing data from 30 individuals of AA broilers. The whole-genome sequencing data is analyzed and compared with publicly available whole-genome sequencing data from 336 individuals of 27 other chicken breeds to obtain 50 SNP sites that differ between the AA broilers and non-AA broilers. The 50 SNP sites are further optimized and analyzed to obtain a combination of 25 SNP sites that exhibit clear breed specificity of the AA broiler. Specific operations are as follows.

Sequencing: the genomic DNA is extracted from blood of the 30 AA broilers, and whole genome resequencing is performed on the 30 individuals of the AA broilers by an ILLUMINA Nova Seq platform (a high-throughput sequencing technology platform of ILLUMINA). An average depth of sequencing reaches 10×, and a total of 262.21 gigabytes (GB) of original sequencing data with a coverage rate of 97.61% (at least 1 base coverage) is obtained.

The whole genome sequencing data of the 336 individuals from the 27 other chicken breeds is downloaded from national center for biotechnology information (NCBI) website. The specific breeds, individual number, and Sequence Read Archive (SRA) accession numbers are shown in Table 3.

TABLE 3
The whole genome sequencing data of the 336 individuals
from the 27 other chicken breeds acquired from NCBI.

| | Individual | |
|---|---|---|
| Breed | number | SRA accession number |
| Red jungle fowl | 36 | SRR1217524, SRR1217525, SRR1217526, SRR1217527, |
| | | SRR1217528, SRR1217529, SRR1217530, SRR1217531, |
| | | SRR1217532, SRR1217533, SRR1217534, ERR2985532, |
| | | ERR2985533, ERR2985534, ERR2985535, ERR2985536, |
| | | ERR2985537, ERR2985538, ERR2985539, ERR2985540, |
| | | ERR2985541, ERR2985542, ERR2985543, ERR2985544, |
| | | ERR2985545, ERR2985546, ERR2985547, ERR2985548, |
| | | ERR2985549, ERR2985550, ERR2985551, ERR2985552, |
| | | ERR2985553, ERR2985554, ERR2985555, ERR298555 |
| Tibetan chicken | 54 | SRR1217491, SRR1217492, SRR1217493, SRR1217494, |
| | | SRR1217495, SRR1217496, SRR1217497, SRR1217498, |
| | | SRR1217499, SRR1217500, SRR1217501, SRR1217502, |
| | | SRR1217503, SRR1217504, SRR1217505, SRR1217506, |
| | | SRR1217507, SRR1217508, SRR3041433, SRR3041434, |
| | | SRR3041435, SRR3041436, SRR3041437, SRR3041444, |
| | | SRR3041445, SRR3041446, SRR3041447, SRR3041448, |
| | | SRR3041449, SRR3041450, SRR3041451, SRR3041452, |
| | | SRR3041453, SRR3041454, SRR3041620, SRR3041692, |
| | | SRR3041713, SRR3041781, SRR3041923, SRR3041924, |
| | | SRR3041925, SRR3041926, SRR3041438, SRR3041439, |
| | | SRR3041440, SRR3041441, SRR3041442, SRR3041443, |
| | | SRR3041455, SRR3041456, SRR3041457, SRR3041458, |
| | | SRR3041504, SRR3041573 |
| Xishuangbanna | 11 | SRR1217509, SRR1217510, SRR1217511, SRR1217512, |
| fighting chicken | | SRR1217513, SRR1217515, SRR1217516, SRR1217517, |
| | | SRR1217518, SRR1217519, SRR1217520 |
| Emei black fowl | 6 | SRR3036337, SRR3041115, SRR3041116, SRR3041121, |
| | | SRR3041122, SRR3041123 |
| Jiuyuan black fowl | 5 | SRR3041124, SRR3041125, SRR3041126, SRR3041127, |
| | | SRR3041128 |
| Jinyang Silky | 6 | SRR3041129, SRR3041130, SRR3041131, SRR3041132, |
| chicken | | SRR3041133, SRR3041134 |
| Muchuan black- | 5 | SRR3041135, SRR3041136, SRR3041137, SRR3041138, |
| bone chicken | | SRR3041364 |
| Miyi chicken | 5 | SRR3036360, SRR3041409, SRR3041410, SRR3041411, |
| | | SRR3041412 |
| Pengxian yellow | 6 | SRR3041413, SRR3041414, SRR3041415, SRR3041416, |
| chicken | | SRR3041417, SRR3041418 |
| Shimian caoke | 4 | SRR3041419, SRR3041420, SRR3041421, SRR3041422 |
| chicken | | |
| Tianfu black-bone | 5 | SRR3041423, SRR3041425, SRR3041426, SRR3041427, |
| fowl | | SRR3041428 |
| Ningdu yellow | 10 | SRR7613951, SRR7613952, SRR7613953, SRR7613954, |
| chicken | | SRR7613955, SRR7613956, SRR7613957, SRR7613958, |
| | | SRR7613959, SRR7613960 |
| Jianghan chicken | 10 | SRR7613961, SRR7613962, SRR7613963, SRR7613964, |
| | | SRR7613965, SRR7613966, SRR7613967, SRR7613968, |
| | | SRR7613969, SRR7613970 |
| Wenchang chicken | 10 | SRR7613971, SRR7613972, SRR7613973, SRR7613974, |
| | | SRR7613975, SRR7613976, SRR7613977, SRR7613978, |
| | | SRR7613979, SRR7613980 |
| Guangxi three- | 10 | SRR7613981, SRR7613982, SRR7613983, SRR7613984, |
| yellow chicken | | SRR7613985, SRR7613986, SRR7613987, SRR7613988, |
| | | SRR7613989, SRR7613990 |
| Huiyang bearded | 10 | SRR7613991, SRR7613992, SRR7613993, SRR7613994, |
| chicken | | SRR7613995, SRR7613996, SRR7613997, SRR7613998, |
| | | SRR7613999, SRR7614000 |
| Huang Lang | 10 | SRR7614001, SRR7614002, SRR7614003, SRR7614004, |
| chicken | | SRR7614005, SRR7614006, SRR7614007, SRR7614008, |
| | | SRR7614009, SRR7614010 |
| Hetian chicken | 10 | SRR7614011, SRR7614012, SRR7614013, SRR7614014, |
| | | SRR7614015, SRR7614016, SRR7614017, SRR7614018, |
| | | SRR7614019, SRR7614020 |
| Huaixiang chicken | 10 | SRR7614021, SRR7614022, SRR7614023, SRR7614024, |
| | | SRR7614025, SRR7614026, SRR7614027, SRR7614028, |
| | | SRR7614029, SRR7614030 |
| Huaibei partridge | 10 | SRR7614031, SRR7614032, SRR7614033, SRR7614034, |
| chicken | | SRR7614035, SRR7614036, SRR7614037, SRR7614038, |
| | | SRR7614039, SRR7614040 |
| Zhengyang three- | 10 | SRR7614041, SRR7614042, SRR7614043, SRR7614044, |
| yellow chicken | | SRR7614045, SRR7614046, SRR7614047, SRR7614048, |
| | | SRR7614049, SRR7614050 |
| Wuhua three- | 10 | SRR7614051, SRR7614052, SRR7614053, SRR7614054, |
| yellow chicken | | SRR7614055, SRR7614056, SRR7614057, SRR7614058, |
| | | SRR7614059, SRR7614060 |
| Xichuan black- | 5 | SRR12103809, SRR12103810, SRR12103811, SRR12103812, |
| bone chicken | | SRR12103813 |
| Commercial | 19 | ERR2985567, ERR2985568, ERR2985569, ERR2985570, |
| broiler A series | | ERR2985571, ERR2985572, ERR2985573, ERR2985575, |
| (India) | | ERR2985576, ERR2985577, ERR2985578, ERR2985579, |
| | | ERR2985580, ERR2985581, ERR2985582, ERR2985583, |
| | | ERR2985584, ERR2985585, ERR2985586 |
| Commercial | 18 | ERR2985587, ERR2985588, ERR2985589, ERR2985590, |
| broiler B series | | ERR2985591, ERR2985592, ERR2985594, ERR2985595, |
| (India) | | ERR2985596, ERR2985597, ERR2985598, ERR2985599, |
| | | ERR2985600, ERR2985601, ERR2985602, ERR2985604, |
| | | ERR2985605, ERR2985606 |
| White leghorn egg | 19 | ERR2985607, ERR2985608, ERR2985609, ERR2985610, |
| chicken | | ERR2985611, ERR2985612, ERR2985614, ERR2985615, |
| | | ERR2985616, ERR2985617, ERR2985618, ERR2985619, |
| | | ERR2985620, ERR2985621, ERR2985622, ERR2985624, |
| | | ERR2985625, ERR2985626, ERR2985629 |
| Rhode Island red | 22 | ERR2985632, ERR2985633, ERR2985635, ERR2985636, |
| chicken | | ERR2985637, ERR2985638, ERR2985639, ERR2985640, |
| | | ERR2985642, ERR2985643, ERR2985644, ERR2985645, |
| | | ERR2985646, ERR2985647, ERR2985648, ERR2985649, |
| | | ERR2985650, ERR2985651, ERR2985652, ERR2985653, |
| | | ERR2985655, ERR2985656 |

Data quality control and filtering: fastp software is used to merge and control quality of the whole genome sequencing data of the 30 AA broilers which is unprocessed and the whole genome sequencing data of the 336 individuals from the 27 other breeds of chickens obtained from NCBI. High quality data is ensured through splicing and removal of low-quality nucleotides, unknown nucleotides (NS), and reads containing over 10% NS.

Analysis and comparison: filtered reads of all individuals are compared with the chicken reference whole genome standard sequence (version number: GRCg7b) by using Burrow-Wheeler aligner software (BWA, version 0.7.17). Sambamba software is used to discard duplicates and remove unmapped or low-mapping quality score reads from comparison results, remaining reads are defined as good reads and used for further analysis, with all parameters using default settings. Genome analysis toolkit (GATK, version 4.0.3.0) is used for SNP calling and the Variant Filtration module is used for filtering. Filtering parameters are set to “QD<2.0‘, ‘QUAL<30.0’, ‘FS>60.0’, ‘MQ<40.0’--cluster-window-size 5-cluster-size 2”, which means that points, with a variation quality/depth ratio less than 2.0, a quality value less than 30, a P-value converted from Fisher test greater than 60, a root mean square of a read comparison quality value less than 40 and a variant number greater than 2 in a 5 base pairs (bp) window, are filtered out. 50 SNP sites that distinguish between AA broiler chickens and non-AA broiler chickens are obtained. Location information of the 50 SNP sites on chicken genome is as follows:

NC_052572.1.70865596,
NC_052572.1.70866183,
NC_052572.1.70881246,
NC_052572.1.70893429,
NC_052572.1.70894238,
NC_052572.1.70886571,
NC_052572.1.85904837,
NC_052572.1.10525401,
NC_052546.1.772828,
NC_052546.1.601275,
NC_052546.1.4650059,
NC_052553.1.594633,
NC_052553.1.3944064,
NC_052541.1.4669367,
NC_052541.1.4864127,
NC_052541.1.5135554,
NC_052541.1.5254806,
NC_052541.1.4633170,
NC_052541.1.4669367,
NC_052572.1.10910679,
NC_052572.1.12398194,
NC_052572.1.53321700,
NC_052572.1.83262553,
NC_052550.1.5129655,
NC_052532.1.82042978,
NC_052557.1.2763553,
NC_052555.1.594633,
NC_052546.1.4864127,
NC_052546.1.10525401,
NC_052546.1.11161858,
NC_052536.1.11174102,
NC_052551.1.13425197,
NC_052551.1.13438787,
NC_052551.1.13448044,
NC_052551.1.13578084,
NC_052551.1.13902189,
NC_052551.1.14131279,
NC_052536.1.11109988,
NC_052536.1.11161858,
NC_052544.1.7566196,
NC_052532.1.120589548,
NC_052532.1.2223786,
NC_052532.1.4650059,
NC_052535.1.873526,
NC_052535.1.963782,
NC_052535.1.3628194,
NC_052536.1.12561592,
NC_052536.1.57411559,
NC_052549.1.8580169.

The version number of the chicken reference whole genome standard sequence is GCA_016699485.1 bGalGal1.mat.broiler.GRCg7b.

Further screening: features of the 50 SNP sites are used as classification features (independent variables) to ensure that a training set obtained through Bootstrap resampling contains data for each SNP. A random forest algorithm and a R language package random forest are used to construct a classification model. Parameters are set as follows: the number of trees (ntree) is 1000, a variable number selected for each branch (mtry) is 4, a proximity matrix is calculated, and other parameters are default. Model generalization ability is evaluated by using an average out-of-bag (OOB) misjudgment rate. A MDSlot function is used to output three-dimensional coordinate data generated by the standardized proximity matrix, and a rgl package is used to draw the sample distribution map in three-dimensional space, graphically displaying the classification effect. A predict function is used to identify varieties, the parameter is set: type=“prob”, and an estimate of accuracy of each identification result is output. The SNP molecular marker combination finally optimized including 25 SNP molecular markers (or sites) has apparent species specificity for the AA broiler. Genotype information for identifying the SNP molecular markers in the AA broiler is shown in Table 1 of the specification. Position and polymorphism information of the SNP molecular markers 1 to 25 of the SNP molecular marker combination in the chicken genome are shown in Table 4.

TABLE 4
specific positions and deoxyribonucleotide information of SNP sites

| SNP molecular | | Specific | |
|---|---|---|---|
| marker | Chromosome | position | deoxyribonucleotide |
| 1 | Z | 70865596 | A or T |
| 2 | Z | 70866183 | T or C |
| 3 | Z | 70893429 | C or G |
| 4 | Z | 70894238 | G or A |
| 5 | Z | 85904837 | C or T |
| 6 | 15 | 4650059 | G or A |
| 7 | 22 | 3944064 | G or A |
| 8 | 10 | 4633170 | A or T |
| 9 | 10 | 4669367 | G or A |
| 10 | 19 | 5129655 | G or A |
| 11 | 1 | 82042978 | C or G |
| 12 | 36 | 2763553 | C or T |
| 13 | 24 | 594633 | A or G |
| 14 | 15 | 4864127 | A or C |
| 15 | 5 | 11174102 | A or G |
| 16 | 20 | 13438787 | A or G |
| 17 | 20 | 13448044 | C or T |
| 18 | 20 | 13578084 | G or A |
| 19 | 20 | 14131279 | A or G |
| 20 | 5 | 11109988 | C or T |
| 21 | 5 | 11161858 | A or G |
| 22 | 13 | 7566196 | T or C |
| 23 | 1 | 120589548 | G or A |
| 24 | 5 | 57411559 | G or A |
| 25 | 18 | 8580169 | C or A |

### Experimental Embodiment 2: Blind Test for Identification Accuracy of the 25 SNP Molecular Marker Combination

In the experimental embodiment 2, the detection kit is used to detect genotypes of 25 SNP molecular markers of 378 unknown chicken samples. The genotypes of the 25 SNP molecular markers are used to identify whether an unknown chicken sample is the AA broiler. Specific operations are as follows.

Samples to be tested: AA broiler, Hubbard broiler, Kebao broiler, Gushi chicken, Xichuan black-bone chicken, Lushi green-shell egg chicken, Fufeng partridge chicken, Guifei chicken, and Hyline chicken, 9 breeds with 378 chicken individuals.

The experiment method includes following steps.

### (1) Genomic DNA Extraction and PCR Amplification

Blood of the 378 chicken individuals from the 9 breeds is extracted, followed by extracting genomic DNA, the PCR amplification is performed to 25 SNP sites by the detection kit. The PCR system for KASP amplification is shown in Table 5.

TABLE 5
the PCR system for the KASP amplification
(10 microliters, abbreviated as μL)

| Composition | Addition |
|---|---|
| 2 × KASP Master Mix | 5 μL |
| primer mix | 2.5 μL |
| Template DNA (10-20 ng/μL) | 2.5 μL |

Reaction conditions are as follows: 94° C. for 15 minutes; 94° C. for 20 seconds, 61° C. for 60 seconds, descending at a rate of 0.6° C./cycle for 10 cycles; 94° C. for 20 seconds, 55° C. for 60 seconds, and 26 cycles. If no fluorescence signal is detected at the end of an initial reaction, additional steps can be added: 94° C. for 20 seconds, 57° C. for 60 seconds, and 3 cycles.

Note: reaction parameters in a reaction program can be adjusted appropriately according to different PCR amplification instrument models, enzymes and primers etc.

### (2) Fluorescence Signals and Genotyping

PCR amplification products are detected by using a platform capable of detecting FAM and VIC fluorescence wavelengths, and then examined with a fluorescence microplate reader. Then, the SNP viewer 2.0 software developed by laboratory of the government chemist (LGC) company is used to read detection data, and SNP genotyping is performed based on the fluorescence signal ratio to obtain genotype information for 25 SNP sites of each chicken individual.

### (3) Comparison

The genotype information of the 25 SNP sites of each chicken individual is compared with the genotypes of the 25 SNP sites listed in Table 1. Those that meet criteria are identified as the AA broilers. The identification results are then compared with the actual chicken breeds to verify the accuracy of the identification.

Experiment results: in the blind test, a total of 50 AA broilers and 328 chickens of 7 other breeds are identified. By comparing the identification results with actual breeds, the identification accuracy is calculated to be 99.47%. Detailed results are shown in Table 6.

TABLE 6
the identification accuracy of blind test samples by 25 SNP sites

| | Individual | Accurately | Accuracy |
|---|---|---|---|
| Breed | number | identified number | rate % |
| AA broiler | 50 | 50 | 100 |
| Hubbard broiler | 50 | 50 | 100 |
| Kebao broiler | 38 | 36 | 94.73 |
| Gushi chicken | 50 | 50 | 100 |
| Xichuan black-bone | 50 | 50 | 100 |
| chicken | | | |
| Lushi green-shell egg | 50 | 50 | 100 |
| chicken | | | |
| Fufeng partridge | 30 | 30 | 100 |
| chicken | | | |
| Guifei chicken | 30 | 30 | 100 |
| Hyline chicken | 30 | 30 | 100 |
| Total | 378 | 376 | 99.47 |

In summary, in the disclosure, the whole genome sequencing data of 30 AA broilers is obtained through whole genome resequencing. By comparing and analyzing the whole genome sequencing data of 30 AA broilers with the whole genome sequencing data of 336 individuals from 27 other publicly available chicken breeds, the 50 distinct SNP sites are identified for the AA broilers and the non-AA broilers. Furthermore, the 25 SNP sites are obtained through combination optimization validation by using the random forest algorithm to form the SNP molecular marker combination for identifying the AA broiler. Moreover, the random forest algorithm can effectively consider interrelationships between various SNP sites, make the features of each site correlated and improve the accuracy of AA broiler breed identification. The disclosure achieves an accuracy rate of 99.47% in testing.

Finally, it should be noted that the above embodiments are only used to illustrate the technical solution of the disclosure, and not to limit it. Although the disclosure is described in detail with reference to the embodiments, those skilled in the art should understand that they can still modify the technical solutions recorded in the embodiments, or equivalently replace some or all of the technical features. The modifications or replacements do not make the essence of the corresponding technical solutions deviate from the scope of the technical solutions of the various embodiments of the disclosure.

What is claimed is:

1. A single nucleotide polymorphism (SNP) molecular marker combination for identifying an arbor acres (AA) broiler, comprising: 25 SNP molecular markers, wherein SNP sites of the SNP molecular markers 1 to 25 are located at a 51^{st} position of each of nucleotide sequences shown in SEQ ID NO: 1 to 25.

2. A detection kit, comprising: a polymerase chain reaction (PCR) primer set for detecting genotypes of the SNP molecular marker combination for identifying the AA broiler as claimed in claim 1.

3. The detection kit as claimed in claim 2, wherein the PCR primer set is a competitive allele specific PCR (KASP) primer set.

4. The detection kit as claimed in claim 3, wherein the KASP primer set comprises a first primer set to a twenty-fifth primer set correspondingly detecting the SNP molecular markers 1 to 25, and nucleotide sequences of the first primer set to the twenty-fifth primer set are shown as SEQ ID NO: 26 to 100.

5. The detection kit as claimed in claim 2, further comprising: a KASP reaction buffer, deoxyribonucleic acid (DNA) polymerase, and deoxy-ribonucleoside triphosphates (dNTPs).

6. An application method of the SNP molecular marker combination as claimed in claim 1, comprising:
identifying AA broiler germplasm resources by using the SNP molecular marker combination.

7. The application method of the SNP molecular marker combination as claimed in claim 6, comprising:
detecting genotypes of SNP molecular markers 1 to 25 in a sample to be tested, wherein when the genotypes of the SNP molecular markers 1 to 25 of the sample to be tested match target genotypes, the sample to be tested is the AA broiler.

8. An application method of the detection kit as claimed in claim 2, comprising:
identifying AA broiler germplasm resources by using the detection kit.

9. The application method of the detection kit as claimed in claim 8, comprising:
detecting genotypes of SNP molecular markers 1 to 25 in a sample to be tested, wherein when the genotypes of the SNP molecular markers 1 to 25 of the sample to be tested match target genotypes, the sample to be tested is the AA broiler.

10. The application method of the detection kit as claimed in claim 9, wherein the detecting genotypes of SNP molecular markers 1 to 25 in a sample to be tested, comprises:
performing PCR amplification reaction by using the detection kit with extracted DNA of the sample to be tested as a template to obtain fluorescence signals for genotyping.
